SEQUENCE LISTING (8 sequences)

SEQ ID NO: 1 - DNA, 1300 bp, Artificial Sequence
  synthetic construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 500 bp, Artificial Sequence
  synthetic construct
cccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccc

SEQ ID NO: 3 - DNA, 70 bp, Artificial Sequence
  synthetic construct
ccacaaacaaacaaccacacaaacacacaaccacaacaccaacacacaaacaaaccaacacacaaactcc

SEQ ID NO: 4 - DNA, 48 bp, Artificial Sequence
  synthetic construct
aaaaaaaaaaaaaaaaaaaaaaaaaaaaaaaaaaaaaaaaaaaaaaaa

SEQ ID NO: 5 - DNA, 196 bp, Artificial Sequence
  synthetic construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - DNA, 48 bp, Artificial Sequence
  synthetic construct
caaacaaaccaacacacaaactcccctcaaacacacaaccaaacaaac

SEQ ID NO: 7 - DNA, 100 bp, Artificial Sequence
  synthetic construct
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 87 bp, Artificial Sequence
  synthetic construct
ccacaaacaaacaaccacacaaacacacaaccacaacaccaacacacaaacaaaccaacacacaaactcctatagtgagtcgtatta

(19) United States
(12) Patent Application Publication
(10) Pub. No.: US 2004/0110205 A1
(43) Pub. Date: Jun. 10, 2004

(54) Methods and systems for nanopore data analysis

(76) Inventor: Hui Wang, Palo Alto, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/668,749

(22) Filed: Sep. 23, 2003

Related U.S. Application Data
(60) Provisional application No. 60/412,959, filed on Sep. 23, 2002.

(51) Int. Cl. C12Q 1/68; G06F 19/00; G01N 33/48; G01N 33/50

(52) U.S. Cl. 435/6; 702/20

(57) ABSTRACT

Systems and methods of performing nanopore data analysis are provided. A representative system includes a nanopore system. The nanopore data analysis system includes a nanopore device and a nanopore data analysis system. The nanopore device includes a structure having an aperture therethrough. The nanopore data analysis system is operative to: generate nanopore data points corresponding to each target polymer and each non-target polymer traversing the aperture of the nanopore structure; form a distribution pattern of the data points; and analyze the distribution of target polymer data points in the distribution pattern.

### CROSS-REFERENCE TO RELATED APPLICATION

This application claims priority to copending U.S. provisional application entitled, “Assessment of Nucleic Acids with a Nanopore,” having serial No. 60/412,959, filed Sep. 23, 2002, which is entirely incorporated herein by reference.

### BACKGROUND

Nanopore technology is one method of rapidly detecting nucleic acid molecules. The concept of nanopore sequencing is based on the property of physically sensing the individual nucleotides (or physical changes in the environment of the nucleotides (i.e., electric current)) within an individual polynucleotide (e.g., DNA and RNA) as it traverses through a nanopore aperture. The use of membrane channels to characterize polynucleotides as the molecules pass through a small ion channel has been studied by Kasianowicz et al. (Proc. Natl. Acad. Sci. USA. 93:13770-3, 1996, incorporate herein by reference) by using an electric field to force single-stranded RNA and DNA molecules through a 2.6 nanometer diameter nanopore aperture (i.e., ion channel) in a lipid bilayer membrane. The diameter of the nanopore aperture permitted only a single strand of a polynucleotide to traverse the nanopore aperture at any given time. As the polynucleotide traversed the nanopore aperture, the polynucleotide partially blocked the nanopore aperture, resulting in a transient decrease of ionic current. Since the length of the decrease in current is directly proportional to the length of the polynucleotide, Kasianowicz et al. were able to determine experimentally lengths of polynucleotides by measuring changes in the ionic current.

The purity and chemical integrity of nucleic acid preparations impact the efficiency of key biomolecular interactions such as nucleic acid hybridization, enzymatic reactions, and chemical modifications. Consequently, purity and chemical integrity can limit the accuracy and reliability of routine molecular biology and biochemistry investigations as well as the expanding field of array technologies. While traditional techniques such as electrophoresis, HPLC, FPLC, and mass spectrometry can assess DNA or RNA sample purity and chemical integrity, the sensitivity of these methods is limited by the relative size and quantity of contaminating nucleic acids. More importantly, the resolution of these methods decreases with increasing DNA or RNA length. Sample evaluation is difficult for nucleic acids with over 100 nucleotides and is virtually impossible for those over 1000 nucleotides.

### SUMMARY

Systems and methods for performing nanopore data analysis are provided. A representative embodiment of a system includes a nanopore system. The nanopore system includes a nanopore device and a nanopore data analysis system. The nanopore device includes a structure having an aperture therethrough. The nanopore data analysis system is operative to: generate nanopore data points corresponding to each target polymer and each non-target polymer traversing the aperture of the nanopore structure; form a distribution pattern of the data points; and analyze a distribution of target polymer data points in the distribution pattern.

One embodiment of the method of performing nanopore data analysis, among others, can be broadly summarized by the following steps: providing a sample including target polymers and non-target polymers and a nanopore device, wherein the target polymers and non-target polymers are selected from polynucleotides and polypeptides; introducing the sample to the nanopore device; generating nanopore data points corresponding to each target polymer and each non-target polymer traversing an aperture of the nanopore; forming a distribution pattern of the nanopore data points; and analyzing a distribution of polymer data points in the distribution pattern.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

Reference is now made to the following drawings. Note that the components in the drawings are not necessarily to scale.

FIG. 1 is a flowchart depicting functionality associated with an embodiment of a polynucleotide analysis system.

FIG. 2 is a flowchart depicting functionality of an embodiment of a nanopore analysis system for assessing length variance and the ratio of target polynucleotides to non-target polynucleotides.

FIGS. 3A through 3C illustrate scatter plots showing that the nanopore analysis system can be used to assess length variance and the ratio of target polynucleotides to non-target polynucleotides.

FIG. 4 illustrates a graph depicting the affect of temperature on nanopore analysis.

FIG. 5 is a flowchart depicting functionality of another embodiment of a nanopore analysis system for assessing target polynucleotide phosphorylation changes.

FIGS. 6A through 6C illustrate scatter plots showing that the nanopore analysis system can be used to assess phosphorylation changes in target polynucleotides.

FIG. 7 is a flowchart depicting functionality of another embodiment of a nanopore analysis system for assessing chemical integrity.

FIGS. 8A through 8E illustrate scatter plots showing that the nanopore analysis system can be used to assess the chemical integrity of a target polynucleotide sample.

### DETAILED DESCRIPTION

As will be described in greater detail here, systems and methods of performing nanopore data analysis are provided. Nanopore analysis systems potentially provide high speed sampling with single-molecule resolution, which may enable unprecedented dynamic range and sensitivity in analysis of samples containing charged polymers such as, but not limited to, polynucleotides and polypeptides. By way of example, some embodiments can be used to determine chemical and/or physical properties of the polynucleotides and/or polypeptides present in a sample as well as the purity of the sample. For instance, the data analysis can be used to identify the chemical states of the polynucleotides as well as the chemical integrity of the polynucleotides. In addition, the data analysis can be used to determine the relative quantity of the components present in the sample.

The term “polynucleotide” refers to nucleic acid polymers or portions thereof such as, but not limited to, oligonucleotides (e.g., up to 100 nucleotide bases), polynucleotides (e.g., greater than 100 nucleotide bases), both of which can be deoxyribonucleotide, ribonucleotide, and/or any natural or synthetic nucleic acid analogs in either single- or double-stranded forms. The term “polypeptide” refers to amino acid polymers or portions thereof such as, but not limited to, proteins and fractions of proteins. For clarity, reference to polynucleotides is made throughout the remainder of this disclosure. However, the methods and systems of this disclosure can be modified and applied to the analysis of polypeptides.

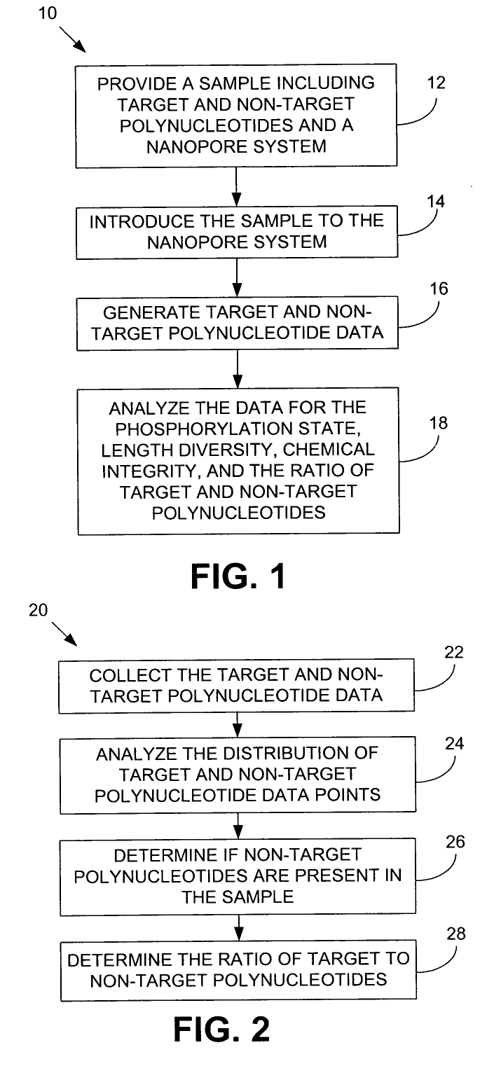

FIG. 1 is a flowchart depicting functionality of an embodiment of a nanopore data analysis system 10 that can be used to analysis nanopore data. As shown in FIG. 1, the functionality (or method) may be construed as beginning at block 12, where a sample and a nanopore system are provided. The sample can include components such as, but not limited to, target polynucleotides (i.e., the polynucleotides of interest) and non-target polynucleotides (i.e., polynucleotide impurities in a sample and/or other impurities in the sample such as target polynucleotides having a guest molecule (e.g., peptide) associated with the target polynucleotide). In general, the sample has been prepared to include one or more specific target polynucleotides, but often contains some contaminant non-target polynucleotides. In block 14, the sample is introduced to the nanopore system. The nanopore system includes, but is not limited to, a nanopore data analysis system and a nanopore device. The nanopore device includes components such as, but not limited to, a nanopore structure that divides the nanopore device into two chambers, wherein one side is a cis chamber and the other side is a trans chamber.

The nanopore structure can include, but is not limited to, solid state nanopore structures or biomolecular nanopore structures. The solid state nanopore structure can be made of materials such as, but not limited to, silicon nitride, silicon oxide, mica, polyimide, and Teflon@. The biomolecule nanopore structures can be made of materials such as, but not limited to, a biomoleucle (e.g., alpha-hemolysin) embedded in a lipid membrane, or a lipid membrane on a solid support.

The nanopore structure can include one or more nanopore apertures. The nanopore aperture can be dimensioned so that only a single-stranded polynucleotide can translocate through the nanopore aperture at a time. For example, the nanopore aperture can have a diameter of about 2 to 4 nanometers (for analysis of single-stranded polynucleotides). In addition, the nanopore structure can include, but is not limited to, detection electrodes and detection integrated circuitry to monitor the translocation of the polynucleotide through the aperture.

In general, the cis and trans chambers include a medium, such as a fluid, that permits adequate polynucleotide mobility for substrate interaction. Typically, the medium is a liquid, usually aqueous solutions or other liquids or solutions, in which the polynucleotides can be distributed. When an electrically conductive medium is used, it can be any medium that is able to carry electrical current. Such solutions generally contain ions as the current-conducting agents (e.g., sodium, potassium, chloride, calcium, cesium, barium, sulfate, or phosphate). Conductance across the nanopore aperture can be determined by measuring the flow of current across the nanopore aperture via the conducting medium. A voltage difference can be imposed across the barrier between the pools using appropriate electronic equipment. Alternatively, an electrochemical gradient may be established by a difference in the ionic composition of the two pools of medium, either with different ions in each pool, or different concentrations of at least one of the ions in the solutions or media of the pools.

The polynucleotides are translocated through the aperture of the nanopore structure by a voltage bias across the nanopore structure to produce an ion current through the aperture. The ion current drives the polynucleotide from the cis side of the nanopore device through the aperture into the trans side of the nanopore device. In general, polynucleotides having different lengths translocate with different duration; the per nucleotide translocation rate is unaltered. The translocation occurs on a microsecond time scale. For example, in minutes, thousands of polynucleotides can translocate through a single aperture by applying 120 millivolts (mV) at temperatures from about 16 to 25° C.

In block 16, nanopore data corresponding to the target and non-target polynucleotides in the sample is generated and collected by the nanopore data analysis system. The translocation of the target and non-target polynucleotides can be expressed using a scatter plot showing each translocation event's normalized average current as a function of that event's corresponding translocation duration. Typically, in a sample having only single-stranded target polynucleotides having no stable base pairing structures, the scatter plot appears as two clusters. The relative positioning of the two clusters is independent of sample concentration or the temperature of the nanopore device. In addition, the cluster patterns can be distinct when the target polynucleotide is relatively short (e.g., about 40 base units long) or long (e.g., greater than 1000 base units long). In some embodiments, the scatter plot distribution does not form a cluster, which may indicate that the sample includes less than a calibration specified fraction of the target polynucleotides.

In block 18, the nanopore data can be analyzed by the nanopore data analysis system to determine the phosphorylation state of a target polynucleotide, length diversity among polynucleotides present in a sample, the chemical integrity of the target polynucleotide, and the ratio of target polynucleotides to non-target polynucleotides in the sample, for example. Additional details regarding each particular analysis are discussed below. In general, the analysis would be conducted on samples having one or more known target polynucleotides. Therefore, analyses as those mentioned above can be important in determining the composition of the sample prior to being used to perform experiments. In addition, the composition of the sample can be important to inspect if the sample has been chemically treated or stored for a length of time, both of which can cause deterioration of the target polynucleotides.

In particular, the nanopore data analysis system can be used to assess the quality of target polynucleotides and the level of backbone fragmentation after chemical synthesis, chemical modification, enzymatic synthesis, and enzymatic modification. For example, the nanopore data analysis system can be used to assess target polynucleotides after: attaching chemical groups for immobilization, attaching chemical groups for chemical linkage, attaching poly-A tail or other specialized nucleic acids, attaching other chemical tags to change translocation signals, protein/enzyme/peptide conjugation, attaching chemical groups for detection or visualization, assessing enzymatic reactions, performing enzymatic reactions such as chemical ligation, site specific probing of nucleic acid conformation, site specific probing of nucleic acid interactions, site specific probing of protein-nucleic acid interactions, probing of none-specific nucleic acid-protein interactions, depurination, depyrimidination, ionization, alkylation, deamination, intercalation, phosphorylation, organic and inorganic extractions, purification procedures, denaturation (e.g., chemical and/or thermal), renaturation (e.g., chemical and/or thermal), interactions with other organic molecules (e.g., carcinogens), interactions with other inorganic molecules, exposure/crosslinking, and/or free radical reactions.

In addition, the nanopore data analysis system can be used to assess the success/failure of modifications to the target polynucleotides that result in changes in translocation profiles. For example, the nanopore analysis system can be used to assess target polynucleotides after: attaching chemical groups for immobilization, attaching chemical groups for chemical linkage, attaching poly-A tail or other specialized nucleic acids, attaching other chemical tags to change translocation signals, protein/enzyme/peptide conjugation, attaching chemical groups for detection or visualization, assessing enzymatic reactions, depurination, depyrimidination, ionization, alkylation, deamination, intercalation, phosphorylation, interactions with other organic molecules (e.g., carcinogens), interactions with other inorganic molecules, and/or UV exposure/cross linking.

Further, the nanopore data analysis system can be used to assess the quality of DNA or RNA bases and level of backbone fragmentation or extension with storage in testing buffers, temperatures, containers, and/or conditions.

Furthermore, the nanopore data analysis system can be used to assess the efficiency of enzymatic reactions in: depurination, deamination, alkylation, depyrimidination, restriction digestion, endonuclease digestion, exonuclease digestion, base excision, transcription, polymerization (e.g., template or non-template directed), efficiency of repair, protein/peptide conjugation, ligation, phosphorylation, methylation, demethylation, and/or acetylation/deacetylation.

Still further, nanopore analysis systems that are solid state structures can be used to assess changes in translocation profile due to local conformational, density and/or charge changes resulting from inter-and/or intra-molecular interactions, such as, but not limited to, detection and/or assessing efficiency of intercalators binding for both site-specific and non-specific interactions, detection and/or assessing efficiency of protein binding for both site-specific and non-specific, UV-crosslinkage, chemical crosslinkage, site specific protein/peptide binding, site specific binding of other organic molecules, and/or site specific binding of antisense tools such as nucleic acid and nucleic acid derivatives.

Typically, the functionality described with respect to FIG. 1 can be implemented, at least in part, in hardware, software, and/or combinations thereof. The nanopore system 10 includes, but is not limited to, equipment capable of measuring characteristics of the polynucleotide as it interacts with the nanopore aperture, a computer system capable of recording the molecular interactions with specific parameters and storing the corresponding data, control equipment capable of controlling the conditions of the nanopore device, and components that are included in the nanopore device that are used to perform the measurements as described below. In addition, the nanopore data analysis system 10 can record signals such as, but not limited to, the amplitude and/or duration of individual conductance and/or electron tunneling current changes across the nanopore aperture.

Functionality of the one aspect of a nanopore data analysis system 20 is depicted in the flowchart of FIG. 2. As shown in FIG. 2, the functionality may be construed as beginning at block 22, where the target and non-target polynucleotide data are collected for a sample. In block 24, the distribution of the target and non-target polynucleotide data points is analyzed. As discussed above, the analysis typically produces a scatter plot having two clusters. In block 26, a determination is made regarding the presence of non-target polynucleotides in the sample. In particular, the presence of non-target polynucleotides in the sample can be determined by observing the data points that are outside of the cluster areas. The cluster areas should contain the data points corresponding to the target polynucleotides since the sample is composed of primarily target polynucleotides. Since polynucleotides having different lengths translocate the aperture with different duration, the target polynucleotides having the same lengths produce data points in the cluster areas, while non-target polynucleotides having a different length than the target polynucleotides produce data points outside of the cluster areas. In addition, non-target polynucleotides having the same length as the target polynucleotide produce data points outside of the cluster areas when the sequence of the non-target polynucleotide and target polynucleotide is not the same.

As mentioned briefly above, the non-target polynucleotides present in the sample can occur as a result of the preparation technique used to produce the target polynucleotides, since techniques such as, but not limited to, enzymatic elongation tend to produce polynucleotides of various lengths. In addition, storage and/or chemical treatment of a sample can lead to deterioration of the target polynucleotides into shorter non-target polynucleotides.

In block 28, a determination is made regarding the ratio of target to non-target polynucleotides. Since the translocation event of each target and non-target polynucleotide is recorded on the scatter plot, a relative ratio of the amount of target to non-target polynucleotide can be determined and as a result, the purity of the sample can be obtained.

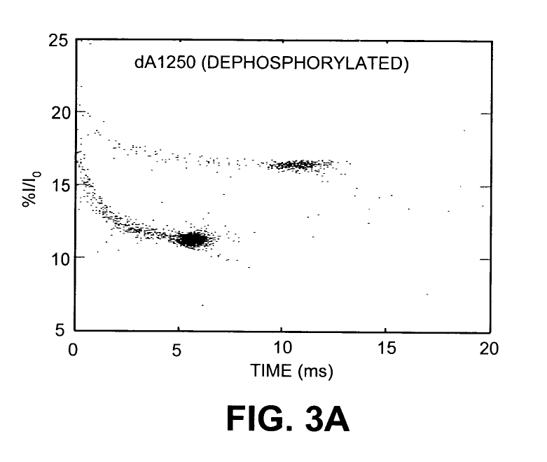

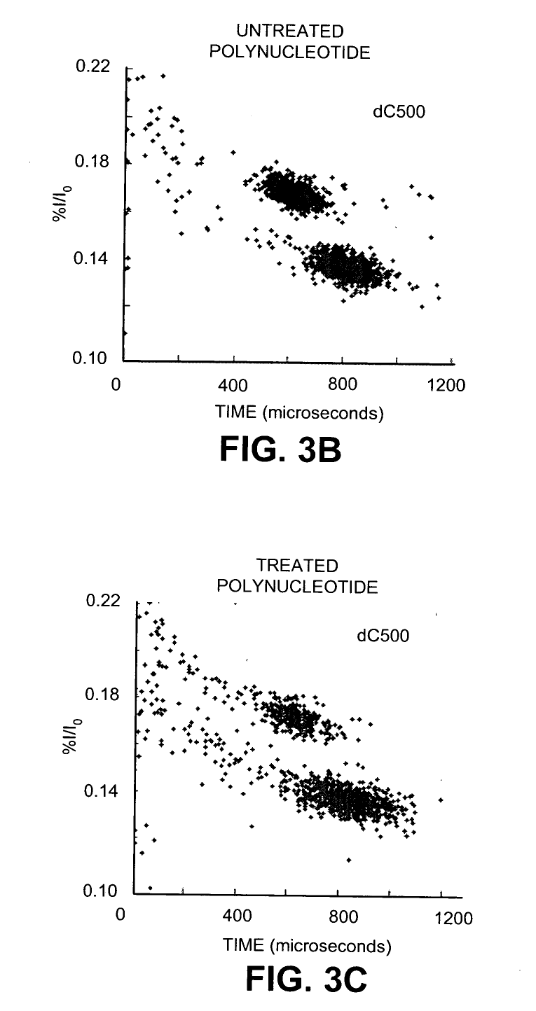

FIGS. 3A through 3C illustrate that embodiments of a nanopore data analysis system can be used to assess the presence of non-target polynucleotides in a sample purportedly having only target polynucleotides (e.g., detect length variance and the ratio of target polynucleotides to non-target polynucleotides). For example, since translocation duration is proportional to the length of target polynucleotide, data points outside of the target polynucleotide clusters can reveal length variance.

FIG. 3A illustrates an example of this for a commercially prepared adenine homopolymer sample of poly dA₁₃₀₀ (SEQ ID NO:1) at 17° C. Because the sample had been generated by non-specific enzymatic elongation, the product should have diverse lengths. Assays of the poly dA sample (SEQ ID NO: 1) with denaturing PAGE revealed a single broad band corresponding to the single-stranded target polynucleotide with approximately 1300 nucleotides. The analysis revealed this predominant 1300 nucleotide product as well as data points generated by smaller non-target polynucleotides. Non-target polynucleotides as small as 10 nucleotides whose ratio to the target polynucleotide was less than 1:600 are as visible as the target polynucleotides in the sample. Even on purposely overloaded gel electrophoretograms, such scattered minor products are usually invisible because of their large length disparity and low relative quantity. The sensitivity of the nanopore system 10 to low abundance non-target polynucleotides can be easily adjusted in real time by sampling translocations for some additional time to increase the number of sampled polynucleotide from hundreds for example. The ability of the nanopore system 10 to register individual protein-DNA interactions enables quantification of relative species with dynamic range.

In addition, FIGS. 3B and 3C illustrates that degradation and backbone scission can be observed by comparing the translocation profile of freshly prepared target polynucleotide dC₅₀₀ (SEQ ID NO:2) (FIG. 3B) and the same target polynucleotide after extended storage and multiple phenol extractions (FIG. 3C).

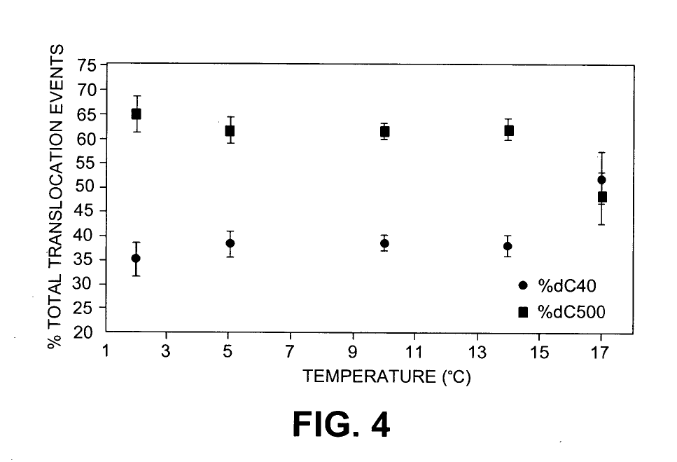

It should also be noted that adjusting the temperature of a nanopore system enhances detection sensitivity towards smaller molecular weight molecules. For example, at temperatures from about 2 to 10° C., there is a bias towards translocating lower molecular weight molecules, as shown in FIG. 4. Thus, a nanopore system can be adjusted to be more sensitive to detecting smaller molecular weight contaminants.

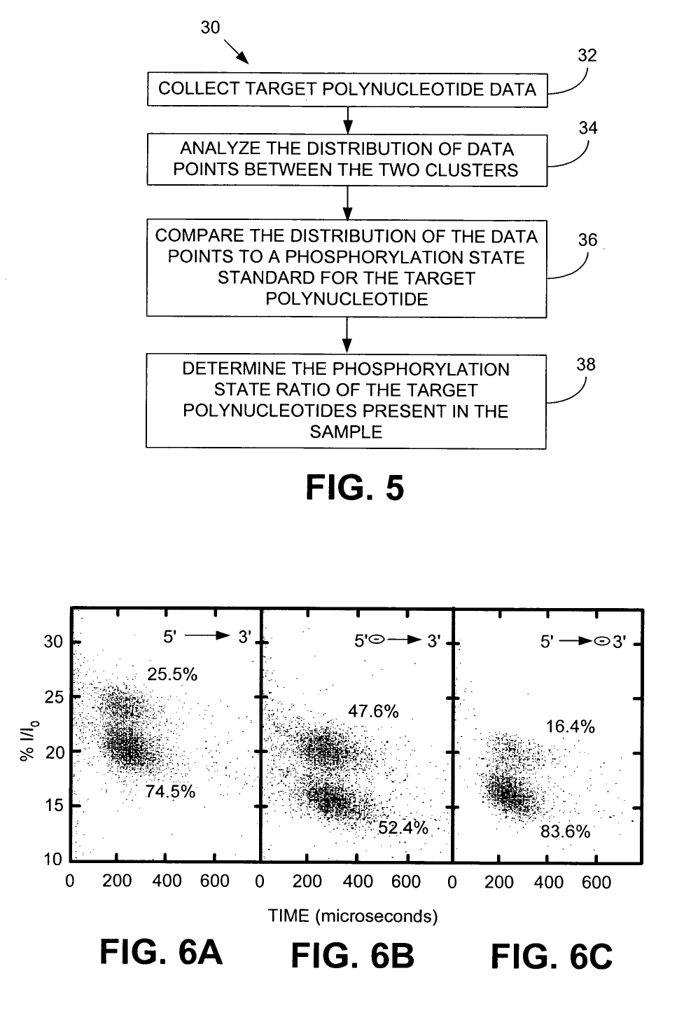

In another embodiment, the functionality of the nanopore data analysis system 30 is depicted in the flowchart of FIG. 5. As shown in FIG. 5, the functionality may be construed as beginning at block 32, where the target polynucleotide data is collected for a sample. In block 34, the distribution of the target polynucleotide data points between the two clusters is analyzed. As mentioned above, one cluster corresponds to the translocation of the target polynucleotide from the 5′ end, while the other cluster corresponds to the translocation of the target polynucleotide from the 3′ end of the polynucleotide. The distribution of the current versus duration data points between the two clusters is a function of the phosphorylation state of the 5′ end and 3′end of the target polynucleotide. For example, the presence of phosphate on the 5′ end of the target polynucleotide, while the 3′ end does not have a phosphate, results in a greater proportion of data points in the cluster corresponding to the 5′ end.

In block 36, the distribution of the target polynucleotide data points is compared to a phosphorylation state distribution standard. The phosphorylation state distribution standard can include scatter plots of one or more distributions between non-phosphorylated and phosphorylated target polynucleotides. For example, the phosphorylation state distribution standard can include distributions from 100% non-phosphorylated and 0% phosphorylated target polynucleotides to 0% non-phosphorylated and 0% phosphorylated target polynucleotides. The specificity of the phosphorylation state distribution standard can be based on the requirements of each particular analysis.

In block 38, the relative amount of target polynucleotides to phosphorylated target polynucleotides can be determined. By comparing the scatter plot of the sample of interest to the phosphorylation state distribution standard, the relative amount of target non-phosphorylated polynucleotides to phosphorylated target polynucleotides can be determined. The precision of the relative amounts depends, in part, upon the phosphorylation state distribution standard. For example, if the phosphorylation state distribution standard only includes one scatter plot of the distribution between the two clusters, then relative ratio of the target polynucleotides to phosphorylated target polynucleotides is less precise than if a plurality of scatter plots of multiple phosphorylation distributions between the two clusters is included in the phosphorylation state distribution standard. As mentioned above, the precision required for a particular analysis can be determined for each analysis.

For example, FIGS. 6A through 6C illustrate target polynucleotide phosphorylation changes in cluster density. In particular, FIG. 6A illustrates a scatter plot of non-phosphorylated target polynucleotide dS₇₀ (SEQ ID NO:3), FIG. 6B illustrates a scatter plot of 5′ phosphorylated target polynucleotide dS₇₀ (SEQ ID NO:3), and FIG. 6C illustrates 3′ phosphorylated target polynucleotide dS₇₀ (SEQ ID NO:3). In FIGS. 6A through 6C, the arrow indicates the 3′ end of the target polynucleotide, while the negative sign “−” denotes phosphorylation.

In FIGS. 6A and 6B, the presence of phosphate on the 5′ end increased the fraction of events in the minor cluster from about 25% for a target polynucleotide bearing no 5′ end phosphate to about 50% for the target polynucleotide bearing 5′ end phosphate. This suggests that the minor cluster represents translocation events initiated by the 5′ end, since the additional negative charge on the phosphorylated 5′ end would likely increase the probability of this end being captured by the electrical bias. The converse is observed in FIG. 6C, where the fraction of events in the major cluster increased from about 75% for a heteropolymer target polynucleotide bearing no 3′ end phosphate to about 82% for the heteropolymer target polynucleotide bearing 3′ end phosphate. Heteropolymer target polynucleotides with both 3′ and 5′ phosphorylation translocated as the 5′ phosphorylated target polynucleotides, with 47% of the events in the minor cluster.

The hypothesis that phosphorylation influences capture probability, and hence translocation direction, is further tested with symmetric molecules. Several different oligonucleotides with either two 3′ ends or two 5′ ends were constructed by linking two 3′ or two 5′ sugar-phosphate backbones of palindromic sequences together with a disulfide bond. As expected, the translocation profiles of the symmetric homopolymers containing either 48 or 196 nucleotides (SEQ ID NO: 4) and (SEQ ID NO: 5) and symmetric heteropolymers containing 48 (SEQ ID NO: 6) nucleotides all exhibited a single cluster positioned at the current values corresponding to the average values of the two clusters observed with equivalent 3′ to 5′ control sequences. Moreover, these cluster positions do not appear to be affected by phosphorylation.

Finally, the nanopore system counted and distinguished between successful and unsuccessful translocation events, the latter exhibiting only partial current blockages that probably represent collisions between polymer and channel or brief polymer visits into only the channel vestibule. The ratio of successful to failed translocation events was therefore compared for the symmetric 3′ ended and 5′ ended molecules. For the 3′ ended symmetric molecule, about 30±10% of translocation attempts failed whereas for the symmetric 5′ ended molecules about 50%±4% failed. Phosphorylation of the 5′ ended molecules reduced the failure rate to about 22%±4%. This suggests that DNA entrance from the 5′ end often fails to translocate and that phosphorylation remedies this problem. This observation accounts for the cluster density bias and illustrates how alterations of cluster densities can reveal phosphorylation.

Embodiments of the nanopore system can be readily used to determine the degree of phosphorylation in a sample using the distribution ratio. Thus, once the distribution ratio is determined for a given target polynucleotide, then the nanopore analysis system can qualitatively determine the phosphorylation state of target polynucleotides in a sample of interest. In general, only a few hundred molecules need to be sampled and the measurement is substantially instantaneous. There is no need for enzymatic analysis or chemical modification of the single stranded target polynucleotide sample and no known length limit for the single stranded target polynucleotide.

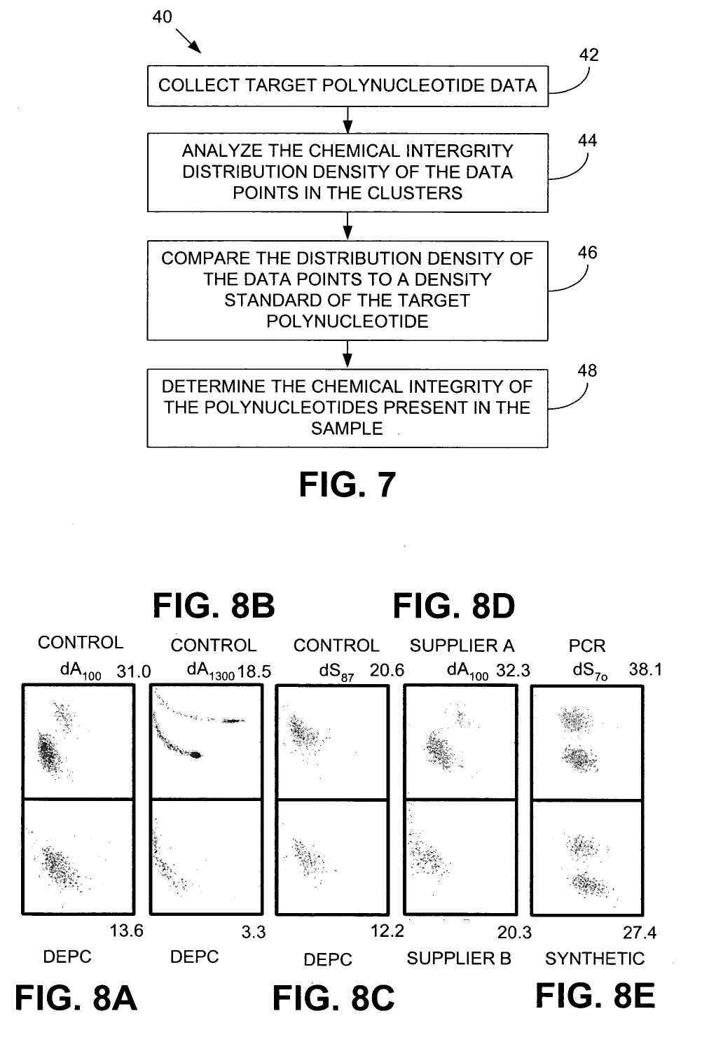

In still another embodiment, the functionality of another nanopore data analysis system 40 is depicted in the flowchart of FIG. 7. As shown in FIG. 7, the functionality may be construed as beginning at block 42, where the target polynucleotide data is collected for a sample. In block 44, distribution density of the target polynucleotide data points in the clusters is analyzed. Since each data point of the translocation profile is generated by the unique interaction between the polynucleotide and the aperture, minor changes in the chemical integrity of the target polynucleotide can affect the electric signals. The changes in chemical integrity can result from chemical treatment of the sample, purification of the sample, and/or storage of the sample, for example.

In block 46, the distribution density of the target polynucleotide data points is compared to a density distribution standard. The distribution density standard can include scatter plots for target polynucleotide samples of one or more samples. In general, the distribution density standard can be used to compare sample distribution densities to determine, for example, the presence of molecular interactions (e.g., base pairing, base aggregation, and adhesion/association of peptides or other small molecules), the affect of chemical treatment of the sample, and the affect of other treatments (e.g., purification, storage, or other handling procedures). For example, chemical modifications to a sample can be assessed by comparing the density distribution before and after chemical modification. In anther example, purification of a sample can be evaluated by comparing the density distribution before and after the purification.

In block 48, the chemical integrity of the target polynucleotides can be determined. By comparing the distribution density of the target polynucleotides in the sample of interest to a density distribution standard, the relative chemical integrity of the target polynucleotides can be determined.

One method of evaluating minor quality differences that can be assessed by a nanopore data analysis system includes using a cluster scoring method to detect target polynucleotide differences. The cluster score for the sample of interest can be compared to the density distribution standard (i.e., cluster score). In addition, cluster scores for a series (e.g., two or more samples) of samples can be compared and ranked. This method works regardless of whether the target molecule translocates as a single cluster or as two clusters as described in the phosphorylation studies above. Therefore, if the cluster score of the sample of interest is similar to the cluster score of the density distribution standard, then the chemical integrity of the sample of interest are similar to the chemical integrity of the standard sample.

In general, the cluster score can be determined for the sample of interest by dividing the scatter plot into arbitrarily selected equal sized areas (e.g., squares or rectangles). The number of data points (translocation events) in each area is counted. The area containing the greatest number of data points is defined as containing a density of 100%. The density of data points in the other areas are defined by the number of data points in each area relative to the area defined as having a density of 100%. Then the total number of data points in the most dense areas (e.g., half, third, or quarter of the most dense areas) is compared to the data points in the least dense areas (e.g., half, third, or quarter of the least dense areas). The ratio of the densest areas to the least dense areas multiplied by 100 is the cluster score. The tighter or more dense the cluster, the higher the cluster score.

One specific example of determining a cluster score includes dividing the scatter plot into rectangular grids of about 20 μsec and 0.2% current units. The data point density for each rectangle is assigned as a percentage of the densest rectangle. The total number of data points in the rectangles with greater than about 50% density is then divided by the total number of data points in the rectangles having less than or equal to about 50% density. Then, the cluster score can be obtained by multiplying the quotient by 100.

FIGS. 8A through 8E illustrate that chemical integrity of a target polynucleotide sample is reflected by its clustering behavior. FIGS. 8A through 8E illustrate five pairs of comparisons, where the cluster score for each scatter plot is displayed in the upper right hand corner of the scatter plot.

For example, the detection of chemical integrity can be illustrated by the translocation profile of dA₁₀₀ (SEQ ID NO: 7) after diethylpyrocarbonate (DEPC) modification. FIG. 8A illustrates that the DEPC-treated target polynucleotide data points are more scattered and contained a greater number of short events than the untreated sample. The treated target polynucleotides generally behaved as though they had difficulty threading through the aperture and exhibited a larger number of very short aborted events, more frequent prolonged blockages, and more variable current blockages than did untreated target polynucleotides. The same effect was observed in homopolymer target polynucleotides as well as heteropolymer target polynucleotides as shown in FIGS. 8A through 8C, SEQ ID NO: 7, SEQ ID NO: 1, SEQ ID NO: 8, respectively. In other experiments (data not shown), translocation profiles of polymers were correlated with their transcription efficiencies before and after DEPC treatment.

To demonstrate applicability of the chemical integrity evaluation, several sets of target polynucleotides are examined. A simple cluster scoring method was applied to objectively evaluate quality differences between samples with identical sequence and length. In the first instance, target polynucleotides obtained from several synthetic DNA suppliers were evaluated. As shown in FIG. 8D, only one of the two suppliers provided target polynucleotides (SEQ ID NO: 7) that translocated through the channel to yield the tightly clustered data points characteristic of high quality target polynucleotides. These samples produced clear, tight, distinct bands when run on denaturing polyacrylamide gels. The target polynucleotides from the other suppliers translocated to produce less clustered scatter plots and appeared as less distinct, somewhat smeared bands in denaturing gel analysis.

The nanopore cluster assay for quality was not confined by specificity of chemical alteration or target polynucleotides size and sequence: target polynucleotides generated by an enzyme in a PCR reaction clustered more tightly than the equivalent chemically synthesized target polynucleotides (SEQ ID NO: 3) from a high quality supplier as shown in FIG. 8E. It is well known that synthesis chemistry and post-synthesis processing can affect polynucleotides base quality, especially for longer polynucleotides. But making the quality distinctions with the nanopore system required fewer tedious manipulations, such as silver staining or radiolabelling, than were required using gels to visualize the few variably degraded polynucleotides in a target polynucleotide sample. While evaluating chemical quality by the polynucleotide band morphology on denaturing gels is constrained by polynucleotide length, the nanopore system has fewer limitations.

Exemplar Experimental Protocol

Nucleic Acid Preparations: Synthetic polynucleotides were purchased from different commercial suppliers. PCR prepared polynucleotides were amplified with synthetic primers from synthetic templates and the synthetic segments were removed from the final products by restriction digests. dA₁₃₀₀ (SEQ ID NO: 1) and dC₅₀₀ (SEQ ID NO: 2) were purchased from Amersham. All DNA except for dA₁₃₀₀ were purified by PAGE under denaturing conditions. PCR products and long homopolymers were generated with 5′ phosphorylation. Most synthetic oligonucleotides were 5′ phosphorylated with phosphoramidite. Dephosphorylation was performed with calf intestine alkaline phosphatase. Some phosphorylations were repeated with T4 polynucleotide kinase. 3′ phosphorylations were performed during synthesis with Glen Research chemical phosphorylation reagent and the unphosphorylated strands were removed with exonuclease I. DEPC reactions were performed at room temperature with 1-5% DEPC with 2 μM DNA for 0.5 to 4 hours. All samples assayed with the nanopore system were also evaluated with denaturing PAGE. The sequence for dS₇₀ (SEQ ID NO:3) was: 5′CCACAAACAAACAACCACACAAACACA CAACCACAACACCAACACACAAACAAACCAACACACAAACTCC 3′ and for dS₈₇ (SEQ ID NO:8): 5′CCACAAACAAACAACCACACAAACACACAAC CACAACACCAACACACAAACAAACCAACACACAAACTCCTATAGTGAGT CGTATTA 3′.

Construction of symmetric molecules: Molecules with two 3′ ends were constructed by oxidation of identical oligonucleotides with deprotected 5′ thiomodifier phoshoramidites. The 5′-ended molecules were constructed with oxidation of oligonucleotides with deprotected 3′ thiomodifier phosphoramidite. The thiomodifier phosphoramidites were supplied by Glen Research. Oxidation products were purified and characterized by denaturing PAGE. Sequences of symmetric 48 mers were either dA homopolymers (SEQ ID NO: 4) or CAAACAAACCAACACAC AAACTCC(-S-S-)CCTCAAACACACAACCAAACAAAC (SEQ ID NO: 6) where S—S indicates disulfide bonds. The control oligonucleotide had the same sequence but did not contain disulfide bonds. Phosphorylations were performed with T4 polynucleotide kinase.

Nanopore set-up and Data Acquisition: Single channel formation, instrument setup and data acquisition was as previously described in Meller, A., et al., Proc. Natl. Acad. Sci. U.S.A. 97, 1079-1084 (2000), which is incorporated herein by reference. All experiments were performed in 1M KCl, 10 mM Tris-HCl pH8 at 25° C., 1 mM EDTA at 2 μsec sampling rate. A 120 mV bias was applied across the channel at 17° C. unless otherwise specified. The amplified signals were low-pass filtered at 100 KHz.

Data Analysis: The software data analysis, implemented in MATLAB R12, consisted of three stages: pre-processing, event extraction, and post-processing. During the pre-processing stage, the experimental data was read from Axon binary files into a data array, and then smoothed with a Daubechies wavelet filter. After all possible translocation events were extracted, the post-processing step tagged and discarded the undesirable events. Using an experienced human eye to examine the current trace from many translocation events, the software was developed to minimize either its accepting unreasonable signals as translocation events or rejecting true translocation events. Cluster scores were calculated as a function of data point density as described above.

It should be emphasized that many variations and modifications may be made to the above-described embodiments. For example, any combination of the nanopore analysis systems 34a, 34b, and 34c can be performed on a sample. All such modifications and variations are intended to be included herein within the scope of this disclosure and protected by the following claims.

We claim at least the following:

1. A method of performing nanopore data analysis, comprising:
providing a sample including target polymers and non-target polymers and a nanopore device, wherein the target polymers and non-target polymers are selected from polynucleotides and polypeptides;
introducing the sample to the nanopore device;
generating nanopore data points corresponding to each target polymer and each non-target polymer traversing an aperture of the nanopore;
forming a distribution pattern of the nanopore data points; and
analyzing a distribution of polymer data points in the distribution pattern.

2. The method of claim 1, wherein the distribution pattern includes at least one data cluster, and wherein analyzing includes analyzing the distribution of target polynucleotide data points within the at least one data cluster.

3. The method of claim 2, further comprising:
comparing the distribution of the target polynucleotide data points between two data clusters to a phosphorylation state standard distribution.

4. The method of claim 3, further comprising:
determining a ratio of phosphorylated target polynucleotide to non-phosphorylated target polynucleotides.

5. The method of claim 2, further comprising:
determining a ratio of phosphorylated target polynucleotide to non-phosphorylated target polynucleotides.

6. The method of claim 1, further comprising:
comparing a density distribution of the target polynucleotide data points to a chemical integrity standard density distribution, wherein a change in the density distribution of target polynucleotide data points as compared to the chemical integrity standard density distribution indicates that the chemical integrity of the target polynucleotides in the sample is different than a chemical integrity for which the chemical integrity standard density distribution was prepared.

7. The method of claim 6, further comprising:
determining the density of target polynucleotide data points in a defined area; and
comparing the density of the target polynucleotide data points to a chemical integrity standard density distribution for the defined area.

8. The method of claim 6, further comprising:
determining the density of target polynucleotide data points in a defined area;
comparing the density of the target polynucleotide data points to a density of the target polynucleotide data points of at least two other samples including target polynucleotides and non-target polynucleotides; and
ranking the samples based on the density of the target polynucleotide data points.

9. The method of claim 6, further comprising:
determining a cluster score for the target polynucleotide data points in a defined area; and
comparing the cluster score for the target polynucleotide data points to a cluster score for a chemical integrity standard density distribution for the defined area.

10. The method of claim 2, further comprising:
analyzing the distribution of the non-target polynucleotide data points.

11. The method of claim 10, wherein distribution of non-target polynucleotide data points outside of the at least one cluster indicates that non-target polynucleotides have a different length than the target polynucleotides.

12. The method of claim 10, wherein distribution of non-target polynucleotide data points outside of the at least one cluster indicates that the non-target polynucleotides have the same length as the target polynucleotide but the sequence of the non-target polynucleotide and target polynucleotide is not the same.

13. The method of claim 10, further comprising:
determining a ratio between the target polynucleotide data points and the non-target polynucleotide data points.

14. The method of claim 1, wherein the failure of polymer data points to form at least one cluster indicates that the target polymers in the sample represent less than a calibration specified fraction of the total polymers in the sample.

15. A system for performing nanopore data analysis, comprising:
a nanopore system including a nanopore device and a nanopore data analysis system, the nanopore device having a structure having an aperture, the nanopore data analysis system operative to:
generate nanopore data points corresponding to each target polymer and each non-target polymer traversing the aperture of the nanopore structure;
form a distribution pattern of the data points; and
analyze a distribution of target polymer data points in the distribution pattern.

16. The system of claim 15, wherein the nanopore data analysis system is further operative to analyze the distribution of the non-target polynucleotide data points.

17. The system of claim 16, wherein the nanopore data analysis system is further operative to determine a ratio between the target polynucleotide data points and the non-target polynucleotide data points.

18. The system of claim 18, wherein the distribution pattern includes at least one data cluster and wherein the nanopore data analysis system is further operative to:
analyze of the distribution of target polynucleotide data points between the two data clusters;
comparing the distribution of the target polynucleotide data points between the two data clusters to a phosphorylation state standard distribution; and
determine a ratio of phosphorylated target polynucleotide to non-phosphorylated target polynucleotides.

19. The system of claim 15, wherein the nanopore data analysis system is further operative to:
determine a cluster score for the target polynucleotide data points in a defined area; and
compare the cluster score for the target polynucleotide data points to a cluster score for a chemical integrity standard density distribution for the defined area in a distribution of a target polynucleotide standard.

20. The system of claim 15, wherein the nanopore data analysis system is stored on a computer-readable medium.

22. The system of claim 15, further comprising:
means for analyzing the distribution of target polynucleotide data points in the distribution pattern
